(19) United States
(12) Patent Application Publication
(10) Pub. No.: US 2026/0224395 A1
(43) Pub. Date: Aug. 6, 2026

(54) MECHANICALLY AIDED FLUID DISPENSING DEVICE

(71) Applicants: Jordan Green, Kent Town (AU); NOVA EYE, INC., Fremont, CA (US)

(72) Inventor: Tony PUGATSCHEW, Kent Town (AU)

(73) Assignee: NOVA EYE, INC., Fremont (CA)

(21) Appl. No.: 19/153,332

(22) PCT Filed: Jul. 26, 2023
(86) PCT No.: PCT/AU2023/050681

§ 371 Date: Aug. 3, 2025

(30) Foreign Application Priority Data
Jul. 27, 2022 (AU) 2022902101
Dec. 5, 2022 (AU) 2022903707

(51) Int. Cl. A61F 9/00

(52) U.S. Cl. CPC A61F 9/0008

(57) ABSTRACT

A fluid dispensing device for dispensing one or more fluids or substances comprising a body that is held by a user. The body comprising a hollow inner section wherein an inner body is located, the inner body adapted to move within the hollow inner section. The inner body capable of protruding and retracting so that once the inner body is pushed forwards to a desired location, a dispensing unit is activated forcing a user defined amount of fluid out of a fluid chamber through a mixing device.

### FIELD OF THE INVENTION

The present invention broadly relates to ophthalmic fluid delivery devices that can deliver various fluid materials such as gases and/or therapeutic liquids. In particular the present invention relates to a device for dispensing fluid(s) and other substances therefrom into the eye of a subject using a delivery/propelled catheter that pushes a user defined amount of fluid into the area of interest in the eye of the subject.

### BACKGROUND TO THE INVENTION

Ophthalmic fluid dispensing devices have been employed to administer various therapeutic fluids into contact with optic tissue including, but not limited to, the sclera, cornea, conjunctiva, and the like. Examples of such devices include eye spray devices, eyedroppers, eye wash units, and the like. Additionally, various ophthalmic devices have been proposed which administer gaseous materials into contact with the eye. Examples of such devices include devices that measure interocular physical characteristics of the eye such as pressure pupil dilation, etc.

There are various types of controlled fluid delivery devices present in the market that allow users to precisely dispense a fluid in a location of interest. Such devices that have been used in the medical industry include, but are not limited to, syringes, robotic control syringes, and mechanically aided syringes.

U.S. Pat. No. 7,201,732 B2 discloses a method and device for administering material to an eye during an eye-open interval. The prior art device includes an electronically controllable fluid delivery element, and a detector in communication with the electronically controllable fluid delivery element. The detector is capable of discerning at least a portion of an eye-blink event and producing a signal actionable on the electronically controllable fluid delivery element.

The following description will focus on the use of the device for injecting fluid into the eye of a person. However, this is by way of example only and the device may be used in animals or in fact for other body parts. In the case of injecting fluid into the eye to assist in for example the treatment of glaucoma, a surgeon or medical practitioner requires precise fluid and volume dispensing. Most of the devices are capable of provide finer precision of dispensing over the traditional syringes but are very difficult for the surgeon to undertake fine precision movements with a syringe to dispense fluid at the same time. As such, this task is commonly a two-person role, requiring a surgical assistant.

Another key drawback is the force that is required for dispensing the various fluids. For example, a high viscous fluid requires a high amount of pressure for dispensing through a small needle or catheter. In most cases, dispensing a high viscous fluid can be sometimes overwhelming for a user while trying to dispense the fluid accurately. To overcome these key issues various types of methods that can force the fluid out of a chamber with mechanical assistance have been implemented. This allows the user to dispense fluid into a regionof interest with mechanical means to overcome the high pressures required for dispensing, ties up valuable resource and adds delays and lack of control as surgeon is dependent on external assistance leading to mistakes.

It is therefore an object of the present invention to provide a fluid dispensing device that overcomes the aforementioned drawbacks, or at least provide the public with a useful alternative.

### SUMMARY OF THE INVENTION

In a first aspect the invention comprises a fluid dispensing device for dispensing one or more fluids or substances, the device comprising a body adapted to be held by a user; the body comprising a hollow inner section wherein an inner body is location, the inner body adapted to move within the hollow inner section; the inner body capable of protruding and retracting; and wherein once the inner body is pushed forwards to a desired location, a dispensing unit is activated forcing a user defined amount of fluid out of a fluid chamber through a mixing device.

In an embodiment, the movement of the inner body is aided by an external actuator means and can be controlled externally.

In an embodiment, the device is adapted to dispense to or more fluids either simultaneously or in succession.

In an embodiment, the device further comprises an energy store that supplies power to the device.

In an embodiment, the fluids are dispensed in a ratio controlled by the size of a nozzle orifice inserted into the device.

In an embodiment, the needle or delivery catheter is adapted to be extended or retracted by the user.

In an embodiment the device further comprises an illumination mechanism.

In an embodiment the device comprises one or more fluid chambers.

In an embodiment the fluid chambers are adapted to contain a storage solution.

In an embodiment the fluid chambers are under vacuum in the fluid chamber.

In an embodiment the devices further comprises a fitting adapted to accept an external fluid chamber.

In an embodiment the device is adapted to dispense the fluid using a mechanical means.

In an embodiment the mechanicals means is pneumatic pressure, a rotational force, an actuator, or a lever.

It should be noted that any one of the aspects mentioned above may include any of the features of any of the other aspects mentioned above and may include any of the features of any of the embodiments described below as appropriate.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows.

FIG. 1 is a side view of the device with the internals visible;

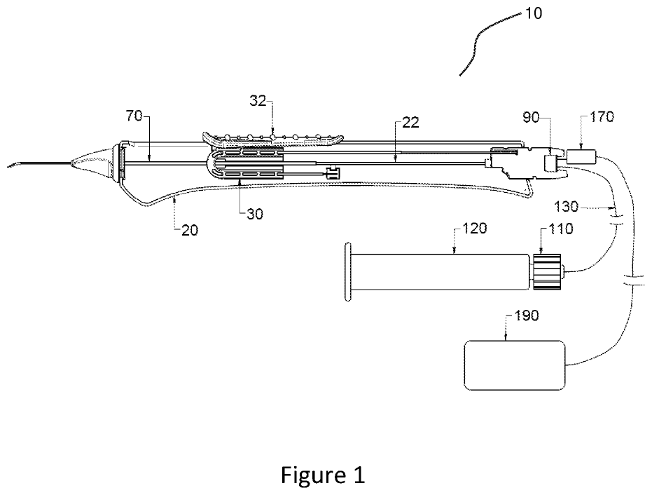

FIG. 2 is a close-up view of the bottom section of the body;

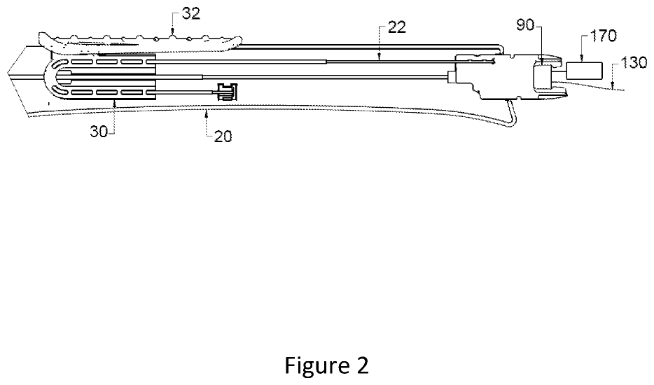

FIG. 3 is a close-up view of the centre section of the body; and

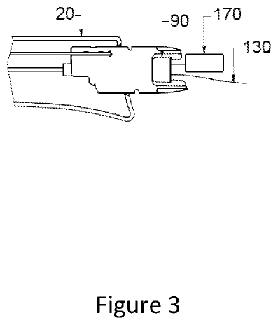

FIG. 4 is a close-up view of the inner body of the device.

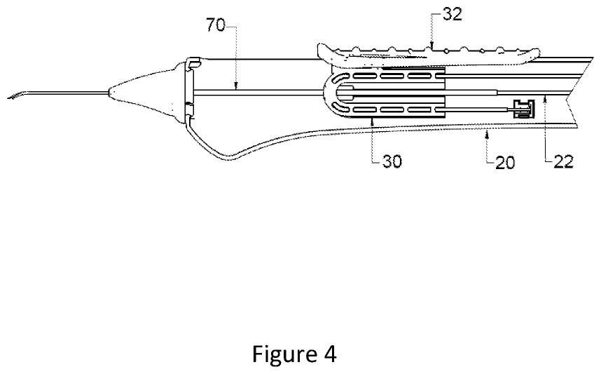

### DETAILED DESCRIPTION OF THE INVENTION

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration.

The device 10 is a handheld device capable of delivering fluid. The device consisting of a body 20, an inner body or catheter 30, connection hub 90, fluid delivery module 110, fluid chamber 120, laser module 170, and a power source 190.

The shape of the body 20 can be, but is not limited to a hand-held device 10 that can be a pistol grip, tube, square, rectangle, oval, T-grip, 1-finger grip, 2-finger grip, sloping, tapered, and D-grip.

The device 10 can also be mounted to a surface and has an arrangement of levers that allow the user to move the device 10 without interference, with the weight of the device 10 fully supported by the mount (not shown). For example, the device 10 can have various arms which are hinged to each other with a mechanical bias between each hinged section. The mechanical bias can be but is not limited to a spring or gas strut. As the device 10 is moved around on the mount, its weight is supported by the mount and maintains the last position it was left by the user without moving. An alternative to a mechanical bias is the use of mechatronic devices that aid the movement of the device 10. These mechatronic devices can be but are not limited to a stepper motor, actuator, servo motor, linear motor, and other electronic motors. The sensors that can be used to detect movement can be but are not limited to load cells, touch sensors, torque sensors, motion sensors, images that are collected and recognise movement through image recognition software packages, toggle sensors, buttons, or any other sensor that can be used to recognise a movement.

The materials used to construct the body 20 can be but are not limited to metal, plastic, ceramics, and carbon fibre. The body 20 can be sterilised using but not limited to gamma radiation, an autoclave, ethylene oxide, formaldehyde, nitrogen dioxide, ozone, hydrogen peroxide, glutaraldehyde, and hypochlorite sterilisations. The length of the body 20 can be but is not limited to 20 mm to 200 mm.

The body 20 consists of an inner body, or catheter 30, the inner body 30 being capable of moving within in the hollow section of the body 22. Movement of the inner body 30 is controlled by the user either by using a mechanical or electronic motion controller. The types of motion controllers can be but are not limited to a rail, actuator, gas, pneumatic, magnetic slide, and a dial.

A rail can be used within the device, wherein the rail is a tab protruding from the hollow section 22 that allows the inner body 30 to slide along. The tab 32 on the inner body 30 allows users to either push or pull on the tab 32 to move the inner body 30. The tab 32 can be connected to a pulley or leverage system wherein once tab 32 moves forward, a lever or pulley amplifies the distance that the inner body 30 moves forward. Such a ratio of the movement of the inner body 30 to the movement of the tab 32 may be but is not limited to a ratio of 5:1, 4:1, 3:1, 2:1 and any other ratio that a user may opt to use. It should be noted that the ratio of movement is not limited and can be adjusted in accordance with user requirements.

An actuator (not shown) can be used to move the inner body 30. The actuator is controlled by the user via a user interface (not shown) that can be located on the body 20 on the device or remote. In the case that the actuator is electrical it can be but is not limited to a range of voltages from 1 to 240V. The actuator is controlled by a microprocessor that is connected to various inputs that can control the movement of the inner body 30. The actuator can also be gas or pneumatic powered wherein a gas or pneumatic fluid is used to move the actuator. As the pressure of the gas or pneumatic fluid is increased, the actuator will move forward thereby moving the inner body 30 forward. When the gas or pneumatic fluid pressure is reduced, the actuator will retract. The pressure can be increased through methods such as but not limited to a user pressing on a compressible flexible vessel, force via a screw, a motor, and a pneumatic pressure machine. For example, as the user compresses the flexible vessel, the pressure is increased, and the actuator is then pushed forward. When the user releases the flexible vessel, the pressure returns to its original pressure and the actuator retracts, pulling the inner body 30 back into its original position. If a piston is used, the piston can be controlled by an electronic motor wherein it pushes a rod forward. As the piston moves forwards it increases the pressure in a chamber and pushes the actuator forward with the inner body 30.

A dial (not shown) can be used to move the inner body 30 wherein the dial is rotated to move the inner body 30. As the user rotates the dial, it moves the inner body 30 in the user's desired direction. The dial can be rotated clockwise or anticlockwise. The shape of the dial can be but is not limited to a circle, oval, cam shape, octagonal, or hexagonal. The edge of the dial can also have various types of surfaces wherein it can be but is not limited to smooth, rigid, toothed, or rough. The surface is configured in accordance with the user's requirement, wherein the user may like to have a surface to have more or less grip. For example, a smooth surface will provide less grip in comparison to a rough surface.

The inner body 30 can be a platform but is not limited to a fluid storage chamber 120, the fluid delivery module 110, power source 190, and an illumination module 170. The inner body 30 can be mounted onto the body 20 wherein it slots into a rail or is retained using bearings.

The fluid storage module 110 can be but is not limited to an internal unit capable (not shown) to receive an external fluid chamber or an internal chamber that can be filled. The internal unit is capable to receive an external fluid chamber wherein the external fluid chamber locks into the internal unit via a fitting. The fittings can comprise of a thread, push lock, cam lock, push-fit, interference fit, flat face, and barbed. As the external fluid chamber is connected to the internal unit chamber, the fluid can be dispensed. The internal unit can also comprise of multiple locations wherein a variety of multiple external fluid chambers can be inserted. For example, the user may want to combine a variety of different fluids for dispensing. The external fluid chambers that are connected to the device can be stored internally or stored externally of the device.

An internal chamber fluid chamber can be used in the device 10 as an alternative or in combination with an external fluid chamber. The fluid can be filled into the fluid chamber 120 using methods such as but not limited to a syringe that is pushed through a seal; a syringe through a port without a seal; and a tube directly into the fluid chamber 120. The device 10 can consist of multiple chambers 120, wherein various fluid chambers 120 can be filled with either the same or different types of fluids. The air is removed from the internal chamber either by but not limited to a channel that prevents air from being trapped, a storage solution, and an air relief valve.

The shape of the internal fluid chamber 120 can be tapered or designed in such a way that when a fluid is inserted into the fluid chamber 120 the air will travel towards the desired location that has an air relief valve that allows air to escape but retains fluid. There is no limitation on the location where the air can escape, it can be positioned in accordance with the user's requirements and other components selected in the device 10.

The fluid chambers 120 can also be prior purged with a fluid, such as but not limited to a storage solution.

The internal fluid chamber (not shown) can also be placed under a vacuum. The internal fluid chamber pulls the fluid into the fluid chamber from the external fluid source via the vacuum in the fluid chamber. This can be used to prevent air entrapment. For example, the fluid chamber 120 is under a vacuum and a syringe with fluid is placed through a seal or connected to a fitting, and once it is connected the fluid is pulled from the syringe into the fluid chamber 120 until the fluid chamber 120 is at atmospheric pressure.

### Dispensing Force

The dispensing from the fluid chambers 120 can be in the form of but not limited to a gas cartridge, press, actuator, foot pedal, or screw. Though this device 10 is not limited to these forms of dispensing force as the device 10 can use any form of method to apply pressure to the cylinders to dispense the fluid from the chambers 120.

### Gas Cartridge

A gas cartridge (not shown) can be used to dispense the fluid in the fluid chambers 120 wherein a gas cylinder can be located internally or externally from the body of the device 10. The shape of the internal gas chamber can be but is not limited to a tube, rectangle, or a custom shape to match the shape of the internal hollow section 22 of the body. An external gas cartridge can be used and connected to the body 20 of the device 10 with a fitting. The fittings used can be but are not limited to a threaded, push lock, cam lock, flat face, and barbed. The gas cartridge can be dispensed using but not limited to a pin piercing a seal, a tube piercing a seal, a valve, and a spring-loaded valve.

The gas that is used within the cartridge can be but is not limited to nitrogen, argon, or oxygen. It is preferable to use a gas that is inert and non-toxic. The pressure of the gas in the chamber can be but is not limited to 16 psi to 2000 psi.

As the gas exits the gas chamber it can flow through to a location wherein it can dispense the fluid, this can be either but not limited to a piston, or compression of the fluid chambers 120.

The gas flow can be controlled with but is not limited to a flow-limiting valve, pressure-reducing valve, cock valve, or the reduction of the orifice size of the tube.

The gas pressure can be controlled with a pressure-reducing device (not shown) wherein it can be in the form of but not limited to a control valve, blow-off valve, and a spring valve with a set pressure. While using the device 10, if the gas cartridge has been depleted, it can be removed and a new gas cartridge can be installed. The new gas cartridge can be installed into the device with a variety of fittings such as an interference fit, threaded, barbed, push lock, push-fit, and flat face. The dispensing of the gas from the gas cartridge can be controlled mechanically and electronically. As an example, the mechanical dispensing can comprise of but is not limited to a lever that controls a pin, when the user presses down on lever, the pin is retracted and allows the gas to pass around the pin. When the lever is released the pin seats back into the gas cartridge opening and sealing it, preventing gas from leaving the gas cartridge. The seal between the pin and the gas cartridge opening can be an interference fit comprising of the following materials plastic, silicon, or rubber seal. The pin can be used in a two-step process wherein the pin firstly goes through an engagement step where the pin is used to pierce into the gas canisters seal.

Once the seal is pierced the gas is capable to escape the gas canister. Once the pin has pierced the seal it then can sit in the gas canister opening to continue sealing the gas canister until the user would like the gas to be released. Alternatively, a hollow needle can be used wherein the needle pierces the gas canister and remains seated in the gas canister, the gas can then travel through the hollow section in the needle through a valve, and the valve is controlled by the user to control the gas discharge. The inner body can be controlled wherein the inner body travels along a hollow tube, the interface between the hollow tube and the inner body is sealed and gas cannot pass through the interface between the two components. Though the two components are capable to move along each other. As the hollow needle travels toward the gas canister, it pierces the gas canister seal, the gas is released through the inner body into the hollow tube. The hollow tube can consist of a flow reducing valve wherein the diameter is reduced through a single or multiple orifice reducer, and can also comprise of a pressure reducing valve wherein a spring-loaded valve releases pressure if the pressure within the hollow tube is above the desired pressure. The inner body can also comprise a one-way valve (not shown). The one-way valve can be located in the hollow tube or within the hollow section around the pin. The one-way valve can allow gas to be dispensed from the gas canister, though preventing gas from moving in the other direction. This allows the user to keep the device under pressure wherein the fluid chamber will be required to retain the same or similar pressure state without the pressure state changing over time as the gas is dispensed. The one-way valve can be but is not limited to a dual disc check valve, swing check valve, lift/piston check valve, ball check valve, silent check valve, nozzle type check valve, wafer check valve, and tilting disc check valve. The tube wherein the gas flows from the gas canister to the fluid chambers can be flexible wherein any pressure transients from the discharge of the gas from the gas canisters are absorbed by the tube's material. The flexiblematerial use can be but is not limited to rubber, silicone, or any other flexible material suitable to retain pressure and flexibility.

As the gas is being dispensed from the gas canister device 10 it can include a gas pressure sensor wherein the user is capable to measure the gas dispensing pressure in real time.

The fluid can be dispensed with a screw, wherein the user will rotate the screw and force the screw towards the fluid chambers 120 to dispense the fluid. The screw can be rotated with a dial or disc, that is attached to the body 20, though is capable to rotate. The screw is held within the disc or dial and can move in response to the movement of the dial or disc. The screw can dispense the fluid either by pushing a piston downwards into the fluid chamber 120 or compressing the fluid chamber 120 to force the fluid out. The screw can be connected to a planetary gearbox, when the screw is rotated it rotates the planetary gearbox, this can move a secondary screw wherein it is geared to a desired ratio for the user. The ratio of the planetary gearbox can be but is not limited to 1:1, 1:2, 1:3, 1:4, 1:5, 1:6, 1:7, 1:8, 1:9, 1:10 or any other desired ratio by the user.

The user can dispense the fluid from the fluid chambers 120 using a mechanical means such as a lever or a button wherein once they press down on the mechanical means the fluid is discharged from the fluid chamber 120. The mechanical means can be in the form of a lever or a button. The lever can be used wherein the user presses down on the lever and a piston is pushed down through the fluid chamber 120 dispensing the fluid from the chamber 120.

An actuator (not shown) can be used to dispense the fluid from the fluid chambers 120. An actuator can be used wherein the actuator will extend outwards in response to a user's request, this may be in the form of a user interface wherein the user's input controls the actuator. The actuator can also be controlled pneumatically where the user has a pneumatic controller that can extend or retract out the actuator's piston. The actuator applies pressure to the fluid chamber 120 to dispense the fluid with a variety of pressure that can be formed by the actuator, the pressure the actuator can force on the fluid chamber can be but is not limited to 15 to 2000 psi.

Any of the devices 10 discussed above can be located inside or externally from the device. If the gas cartridge, dial, press, or actuator are located externally they can be connected to the device with a remote line or tube wherein the force exerted from any of the components is still capable to send to the fluid chamber 120 to dispense the fluid from the fluid chamber 120.

### Multiple Fluid Chamber Dispensing

As the fluid is being dispensed from the fluid chambers 120, the flow can be controlled by the output orifice size. If multiple fluid chambers (not shown) are being used, each of the fluid chambers 120 used can have a variety of different orifice sizes that can be used to control the ratio of dispensing of fluid between each of the fluid chambers 120. The orifice size can also be variable wherein the user can adjust the flow by moving a dial that causes the orifice size to reduce or increase. If there are multiple chambers used, the fluid can be dispensed from each of the fluid chambers sequentially. Dispensing can be controlled by theuser wherein the user selects the mode of dispensing (varied ratio, sequential, or simultaneously) and the fluid chambers will dispense accordingly. For example, if a screw dispenser is used, the dispensing of the fluid between the fluid chambers 120 can be undertaken with a planetary gearbox. If an actuator is being to dispense fluid from the fluid chambers 120, the actuators will extrude in accordance with the electronic or pneumatic controls. For example, the user may state that the movement of one actuator is twice of another actuator, or, one actuator will start and a second one will move once the first actuator has completed moving.

### Fluid Mixing

As the fluid is dispensed from the fluid chamber 120 it travels out of the fluid chamber 120 into a hollow tube 130. The hollow tube can also consist of a mixing apparatus (not shown) wherein the fluids are mixed into each other. The mixing apparatus can comprise of but is not limitedto a static or dynamic mixing component. A static mixing component can comprise of a section wherein the fluid must travel through, as the fluid travels through the static mixing component the flow is disrupted to cause the fluid to mix. The types of static fluid flow components that can be used but are not limited to vanes, cross-sectional rods, meshes, spirals, helical mixers, wafer mixers, counterflow vanes, baffles, beads, and non-flat or reduced diameter tube walls. All these types of static fluid flow mixing components disrupt the fluid flow and may cause the fluid to become turbulent and mix.

As the fluid travels through the tube 130, the fluid will come in contact with the static fluid flow components wherein the flow will be disrupted forcing the fluid to become turbulent within the tube 130. As the fluid is pushed further down the tube 130 the fluid becomes more turbulent causing the fluid to interact with itself even further.

For example, as the fluid is pushed through the tube 130 wherein a helical mixer (not shown) is present, the fluid will follow the helical mixer path causing the fluid to either become turbulent or forcers the fluid to intersect with itself causing it to mix. Another example is if the fluid travels through a tube 130 and there is the presence of mesh wherein the mesh can comprise of a single or multiple meshes, as the fluid travels through the mesh the flow is disrupted and becomes turbulent causing the fluid to mix.

A dynamic fluid flow mixer (not shown) is a form of mixing wherein a rotation element is used to mix the fluids. The dynamic fluid flow mixer can comprise of but is not limited to an impeller, rotating vane, rotating mesh, rotating baffle, rotating helical vane, rotating screw, and a coaxial horizontal baffle.

As the fluid leaves the fluid chamber 120 and interacts with the dynamic fluid flow mixing apparatus the fluid flow becomes disrupted causing turbulence in the tube 130, this helps aid with mixing. As the fluid travels through the dynamic fluid flow mixing component it will rotate due to the force from the fluid force, therefore the mixing is enhanced due to the movement from the dynamic fluid flow mixing apparatus. A device 10 may have one or multiple dynamic fluid flow mixing apparatuses.

At the end of either the static or dynamic mixing components, the tube 130 can contain straighten vanes that can correct the flow of the fluid and straighten the flow before delivery. This can comprise of vanes that are flat and aligned with the final desired flow direction.

The mixing section can be located either within the device 10 or used as an add-on component wherein it is attached to the end of the device 10 via a fitting. The fitting can comprise of but is not limited to a bard, push-fit, inference fit, or threaded component.

The catheter or needle 70 can comprise of a variety of types wherein the needle can be a hypodermic needle, a flexible needle, or a smart needle. A hypodermic needle consists of a typical needle that would be used in the medical industry. The size of the hypodermic needle can comprise of but is not limited to 7 gauge to 34 gauge. The material of the hypodermic needle can comprise of but is not limited to steel, preferable stainless steel. A flexible needle (not shown) can be used wherein the material of the needle 70 can be but is not limited to a flexible material. A flexible needle can also be used wherein the needle is capable to be moved in the desired direction of the user. For example, the user would like the needle to travel around a certain object, and the needle can be controlled by the user to travel around objects of interest. The flexible needle is capable to flex due to a variety of joints that are held together with either flexible joints, hinges, or internal supports.

The needle or catheter 70 can also be connected to a load cell sensor wherein the pressure of the tip is captured. For example, as the user is inserting the needle 70 they are capable to capture the pressure exerted on the top of the needle 70. The pressure can be in relation to the various types of material the needle 70 is being inserted into. Different types of tissues have different types of resistance, therefore the user will be able to visually observe the needle's 70 pressure as it is being inserted and once the pressure drops it may indicate it has passed through a certain layer or region of interest.

The length of the needle or catheter 70 can be but is not limited to 1 mm to 100 mm in length, though preferable the ideal length is between 24 to 45 mm. The needle 70 can also comprise of but is not limited to depth indicators on the needle 70 wherein the user is able to visually track the depth of insertion from the indicators or markings on the needle's 70 outer surface.

The needle or delivery catheter 70 has a hollow section through the centre wherein it can retain the tube 130 for the delivery of fluid, fluid removal (not shown), and optic fibre (not shown) which can be used for illumination. The needle 70 is able to retain any number of tubes and is only limited to the internal diameter. The fibres can be moved within the needle 70 by mechanical means that allow the user to extend or retract the fibres from the needle 70. Forexample, the needle 70 is placed into a patient in an area of interest, the user then extends the fibres 130 further out into the patient by pushing a tab forward wherein the fibres 130 are pushed from inside the needle 70 beyond the needle 70 tip. The mechanical means that are used to extend or retract the fibre from the needle 70 can be but are not limited to a sliding tab, actuator, pneumatic, and screw. For example, the fibres 130 can be mounted to a platform in the body that is capable to shift forward and backwards in the body, the fibres are fed into this platform at a function connection hub 90.

The fluid dispensing device comprises of catheter delivery which may include an energy store that enables the device to be energized. Using the energy store, energy can be stored in different forms such as electric, magnetic etc. although it is preferred to use pressure, e.g. in gas or hydraulic springs, and most preferably as mechanical energy, preferably mechanical springs.

The size of the fibre tube 130 for the fluid delivery can be but is not limited to 10 microns to 1 mm, the preferable size of the fibre tube 130 is to allow it to be enclosed in the needle or catheter 70.

The optical fibre (not shown) is used to illuminate the tip of the needle 70. The optical fibre would commonly use a fibre optic cable wherein the cable used is illuminated with a light source, it must be noted that it is not limited to a fibre optic cable. The light source 170 for the fibre can be the form of but is not limited to a light-emitting diode or a laser diode. The laser duty cycle can be adjusted in accordance with what the user may require. The duty cycle can range from but is not limited to 1 to 100%. For example, a duty cycle of 50% may be used wherein the user has an adequate amount of illumination and the battery life span is increased. The duty cycle can also be changed during the operation of the device 10 wherein an initial duty cycle is used and then followed by a different duty cycle.

The laser 170 can be located internally or externally from the device 10. For example, a source can be located on the device 10 or an external laser source. If the source is located internally the laser diode can be located anywhere in the device 10 and will need an optical fibre connection to the emission side of the laser. If the laser is located externally, a fibre or cable can be used to connect to the device 10. The wavelength of the laser can be but is not limited to 100 nm to 3000 nm, though preferably the use of visible light and UV light. For example, if a user would like to visually see where the needle tip is located in the eye, the user can illuminate the optical fibre allowing the user to visually observe the optical fibre. The user is also capable to select a wavelength that can either excite the fluid that is being dispensed. For example, a fluid that fluoresces can be dispensed and the illumination from the fibre is used to track the dispensed liquid.

The fibre and fluid tube 130 are fed into the needle 70 through a seal, the seal can be but is not limited to silicon, rubber, or any other flexible material. The fibre and fluid tube 130 can run parallel or be intertwined with each other. Intertwining the fluid tube 130 and optical fibre cable can prevent the two tubes from tangling with each other while in the needle 70. Intertwining the cables also prevents one cable from shifting without the other. Other forms of fixing the fibres together can be such as but are not limited to a restraint, knots, and bonding the fibres together.

The laser source 170 can be powered either internally, externally, or inducted. The battery 190 can be but is not limited to a button cell, watch battery, or coin battery. An external power source can also be used wherein the device 10 is connected to an external power cable allowing the user to operate the device 10 with an external power source. The power 190 can also be transferred to the device 10 using wireless power, wherein the power is transferred from the user to the device 10 via a wireless charging board. Though ideally, the device will use an internal source of power such as a battery.

Additional fibres can be introduced wherein the fibre is capable to capture light or a visual image (not shown). For example, a fibre is capable to capture the intensity of fluorescence as it is excited within an area of interest. Another example is the user would like to obtain an image with an optical element attached to the end of the fibre, the fibre is then fed through the seal into the needle 70 wherein an image can be obtained. The optical element can have a diameter less than but not limited to 0.05 mm to 3 mm. The optical element can be arranged so that it is capable of emitting or receiving electromagnetic radiation. The fibre is capable to have various optical elements attached to the end surface of the optical element such as a spherical or aspheric lens, an axicon lens, a Fresnel lens, a total internal reflection lens, a diffractive optical element, and metalenses. The lens may alsobe arranged accordingly for the correction of chromatic, spherical aberration, and astigmatism. An example of use is wherein the user would like to visually observe the discharge of a fluid into a certain section, the user can discharge the fluid through one fibre and observe the discharge of the fluid in another fibre, and the user can remove the fluid through an open fibre.

### User Interface

The device 10 can consist of a user interface (not shown) wherein the user can view all of the sensor data as discussed above. The user interface can be present on the device 10 and/or external. User interfaces that are present on the device 10 can be but are not limited to lights, speakers, buzzers, gauges, and a visual screen.

The lights (not shown) used for the device can be but are not limited to LEDs or laser diodes. The lights can be activated in response to various thresholds that are measured from either of the sensors, for example, the LED can display a variety of colours wherein various variables are linked to those colours and when a variable hits a certain threshold the user can have a visual indication that the device is correctly operating.

A light can also be used to help guide the needle or catheter 70 tip as the user inserts it into a patient. For example, a laser is used to direct the tip of the laser wherein the user would like to pinpoint a location for insertion.

An audible noise can be used as a user interface with the device 10. The audible noise used can be from but is not limited to a speaker, buzzer, or transducer. The noise can be in response to a threshold that has been met in response to sensor data. For example, the user may be dispensing fluid from the device 10, once the device 10 is close to exhausting all of the fluid and audible noise is produced by the device 10. Another example may be wherein the gas canister has not correctly been inserted into the device 10, and the device 10 is unable to dispense fluid.

A gauge (not shown) can be used with the device 10 to indicate to a user a device 10 operational condition. For example, this may be but is not limited to a mechanical or digital gauge. The gauges can provide the user with operational information from the device 10. As an example, the user can visually see the fluid chamber 120 pressure or the volume remaining in each of the fluid chambers 120.

The device 10 can also comprise a visual screen present on the device 10 wherein all of the information in regards to the operational conditions or sensors is displayed on the screen. The visual screen can also comprise of a touch screen wherein thresholds or various other screens can be managed and moved.

The device 10 can also comprise of a communication module that allows it to send sensor data to other devices 10. The methods the device can communicate can be with but are not limited to WIFI, Bluetooth, Zigbee, LoRA, Sigfox, radio, IR, or any other form of communication. It is noted that as communication methods change over time, this device 10 is not limited to what has been listed and is capable to be adapted to any form of communication. For example, the device 10 can collect all of the sensor information on board and then use the communication module to send the data to another location. The external devices can be used to capture the data sent from the communication module wherein the data can be but is not limited to recorded, stored, or displayed.

The device 10 is capable to be programmed wherein using an onboard or external user interface (not shown). The device 10 can be programmed to undertake a fluid dispensing operation. The fluid dispensing operation can consist of a purging operation followed by a fluid dispensing cycle. For example, the device is programmed to recognise once the fluid of interest has been placed into a device 10, the recognition of the fluid is due to the presence of various sensors such as but not limited to pressure, RFID, photoresistor, load cell, voltage change, amperage change, depth sensor, switch, or any other system that can determine the insertion of a fluid chamber 120 into a location of interest. Once the fluid chamber 120 is installed into the device, the device 10 beings to undertake a preprogramed process, wherein fluid will be discharged at a preselected volume to prepare the device for use. Once the program is completed there may be an indication provided by the device 10 to the user. The device 10 is now ready for use wherein the user can dispense the fluid into the location of interest. The fluid can be dispensed with but are not limited to a pre-set volume to be dispensed, fluid is dispensed for a set duration, fluid is discharged in pulse intervals at set volumes, and full manual control. Therefore, the user interface can control the device in all aspects such as but not limited to the movement of the device 10 and discharge of the fluid.

### Example 1

The user firstly holds the device with their hand and inserts a single cartridge of fluid into the fluid chamber 120. The cartridge is threaded into the fluid chamber 120, once the cartridge is connected the piston is seated in the back of the fluid chamber 120. A gas cartridge is then inserted into the back of the device and is locked in place. The device 10 is then primed wherein a lever is pushed down forcing a hollow pin into the gas canister, the gas then travels through the hollow section of the pin past a one-way valve, into the back of the piston forcing it forward and causing the fluid chamber 120 volume to reduce and discharge the fluid. As the fluid is discharged from the fluid chamber 120 it travels through a fibre tube 130 wherein it is intertwined with an optical fibre that is connected to a laser emitting diode. Once the fibre tube 130 has been purged of air and fluid is being discharged from the tip of the needle 70 the user can release the lever. The user is informed by the device that it is ready to use by a green LED flashing, this indicates to the user the device 10 is ready to be inserted and to dispense fluid. The user can then insert the device 10 into a patient such as an eye where the user monitors the pressure of the needle 70 and visually observes the needle tip from the illumination of the fibre in the tip of the needle 70. The user will continue to dispense the fluid from the fluid chamber 120 through the fibre tube 130 that is in the needle 70 until the desired amount of fluid has been dispensed.

### Example 2

The user places the device on a surface and restrains it in place preventing it from moving. The user then fills the fluid chamber 120 by pushing a hypodermic syringe through a seal, once the hypodermic syringe pierces the seal the fluid from the syringe is injected in, the fluid in the syringe is three times the total volume in the fluid chamber 120. As the fluid is pushed into the fluid chamber 120 the storage fluid is discharged out of the end of the needle 70 tip of the device 10. Once there is no more fluid to be dispensed, the user removed the hypodermic needle 70 from the seal and closes the device 10. The user then turns on the laser which illuminates the end of the needle 70. The user then inserts the device into the area of interest in the patient, once the needle 70 is in the location of interest the user can then push the tab forwards pushing the two fibres from inside the needle 70 out further into the area of interest. Once the fibres are in the area of interest the user then pushes down on a lever which forced a piston into the fluid chamber 120 and dispenses the fluid from the fibre 130. Once the fluid is dispensed the user retracts the fibre 130 back into the needle 70 and removed the device 10 from the patient.

### Example 3

The user would like to discharge two types of fluids at a ratio of 1:2. The user then inserts a nozzle in chamber 1 and 2, chamber 1 has half the nozzle orifice size of chamber 2. The user then selects the two fluids of interest that are then screwed into the threaded fittings of the fluid chambers 120. Once the fluid chambers are fitted to the device 10, a piston is placed at the rear of each of the chambers and the device 10 is closed. The device laser diode is then turned on illuminating the end of the needle 70. The user then picks up the device 10 and rotates a dial at the end that rotates a screw that is attached to a planetary gearbox, as the screw rotates in the planetary gearbox it rotates two screws downwards into the rear of the pistons that are held into place by the fluid chambers 120. The fluid chamber 120 will start to dispense fluid and purge the needle 70 as the pistons are forced downwards. Once the fluid is present and coming out the end of the needle 70 the device 10 is ready for use. The fluid leaving the device through the needle is determined by the fluid level sensor and may be considered to be an indicator that the device is primed. The needle 70 is then inserted into the patient wherein thesensor monitors the pressure being exerted onto the tip of the needle 70. The user can also track the needle 70 tip due to the illumination from the optical fibre inside the needle 70.

Once the needle 70 is in the desired location, a tab is pushed towards the needle 70 by the user wherein a fibre is extended out of the end of the needle 70. The fibres extending out from the end of the needle 70 can be visually observed due to the fibre being illuminated. Once the fibres are in the desired location, the user can start to rotate the dials wherein the fluids are dispensed at a ratio of 1:2, this is controlled by the nozzle orifice size that was inserted into the device initially. Once the device 10 has discharged all the fluids into the area of interest, the user obtains a visual cue from a sensor that is not detecting any further flow from the fluid chambers 120. The signal may be in the form of a visual indicator from an LED. The user then retracts the fibres by pulling a tab backwards away from the needle 70, when the fibres have been retracted the user can retract the needle 70 from the patient.

The reader will now appreciate the present invention which provides an improved mechanically aided fluid delivery device.

### LIST OF COMPONENTS

The drawings include the following integers:
10 device
20 body
22 hollow inner section
30 inner body
32 tab
70 catheter or needle
90 function connection hub
110 fluid delivery module
120 fluid storage module
130 fluid fibre tube
170 illumination module
190 power source.

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. A fluid dispensing device for dispensing one or more fluids or substances comprising:
a body adapted to be held by a user;
the body comprising a hollow inner section wherein an inner body is location, the inner body adapted to move within the hollow inner section;
the inner body capable of protruding and retracting; and
wherein once the inner body is pushed forwards to a desired location, a dispensing unit is activated forcing a user defined amount of fluid out of a fluid chamber through a mixing device.

2. The fluid dispensing device as in claim 1, wherein the movement of the inner body is aided by an external actuator means and can be controlled externally.

3. The fluid dispensing device as in claim 1, wherein the device is adapted to dispense two or more fluids either simultaneously or in succession.

4. The fluid dispensing device as in claim 1, wherein the device further comprises an energy store that supplies power to the device.

5. The fluid dispensing device as in claim 3, wherein the fluids are dispensed in a ratio controlled by the size of a nozzle orifice inserted into the device.

6. The fluid dispensing device as in claim 1, wherein the needle or delivery catheter is adapted to be extended or retracted by the user.

7. The fluid dispensing device as in claim 1 further comprising an illumination mechanism.

8. The fluid dispensing device as in claim 1 further comprising one or more fluid chambers.

9. The fluid dispensing device as in claim 8, wherein the fluid chambers are adapted to contain a storage solution.

10. The fluid dispensing device as in claim 8, wherein the fluid chambers are under vacuum in the fluid chamber.

11. The fluid dispensing device as in claim 1, further comprising a fitting adapted to accept an external fluid chamber.

12. The fluid dispensing device as in claim 1, wherein the device is adapted to dispense the fluid using a mechanical means.

13. The fluid dispensing device as in claim 12, wherein the mechanical means is pneumatic pressure.

14. The fluid dispensing device as in claim 12, wherein the mechanical means is a rotational force.

15. The fluid dispensing device as in claim 12, wherein the mechanical means is an actuator.

16. The fluid dispensing device as in claim 12, wherein the mechanical means is a lever.
